(19) United States
(12) Patent Application Publication
(10) Pub. No.: US 2024/0148622 A1
(43) Pub. Date: May 9, 2024

(54) TOPICAL COMPOSITION FOR SKIN

(71) Applicant: Shiseido Company, Ltd., Chuo-ki, Tokyo (JP)

(72) Inventors: Ranran ZHANG, Beijing (CN); Yuling SUN, Beijing (CN); Yoshihito NISHIJIMA, Tokyo (JP)

(73) Assignee: Shiseido Company, Ltd., Chuo-ku, Tokyo, (JP)

(21) Appl. No.: 18/282,327

(22) PCT Filed: Mar. 11, 2022
(86) PCT No.: PCT/CN2022/080429

§ 371 Date: Sep. 15, 2023

(30) Foreign Application Priority Data
Mar. 18, 2021 (CN) 202110292923.9

(51) Int. Cl. A61K 8/25; A61K 8/92; A61Q 1/14

(52) U.S. Cl. CPC A61K 8/25; A61K 8/92; A61Q 1/14; A61K 2800/412

(57) ABSTRACT

The present disclosure relates to a topical composition for skin, which comprises a) a solid particle, which is a porous particle having surface openings, and has an average particle size of primary particles of 0.5 to 20 μm; b) at least one wax; c) at least one carbon chain-based oil; and d) at least one surfactant, and the composition optionally comprises water and has a water content of 5% or less based on the total mass of the composition.

### TECHNICAL FIELD

The present disclosure pertains to the field of cosmetics and specifically relates to a cosmetic for cleansing skin or a composition for removing makeup. In particular, the present disclosure relates to a solid detergent composition containing little or substantially no water.

### BACKGROUND

Compositions for skin cleansing have existed for a long time, which may clean or remove aged cuticles, oil secretion, dirt, cosmetics, or the like from human skin.

Such compositions may generally exist in the form of aqueous compositions such as liquid, semi-solid (e.g., gel). Such conventional personal cleansing and care compositions include makeup removers, facial cleansers, shaving products, etc. In addition, the compositions may also typically comprise one or more ingredients such as essence, preservative, surfactant, etc. In recent years, facial cleansers (including makeup removers) have been the sub-category with the highest penetration rate in the facial skin care market in many markets. Overall, these compositions, in addition to their cleansing function, may also remove moisture from the skin when used, which may cause the skin to feel excessively dry or chapped and may result in a poor user experience.

Besides, there is another kind of composition existing in the form of a solid composition containing little or substantially no water, which, when applied to human skin, may be spread on the skin under external force and cleanse the skin. For example, a type of cleansing product usually referred to as “soft solid” may provide consumers with a soft and dry feeling when applied.

Cited document 1 discloses a solid anhydrous composition comprising hydrophobic aerogel particles, at least one wax with a melting point greater than or equal to 60° C., at least one hydrocarbon-based oil, and at least one nonionic surfactant. The composition has improved makeup remove ability, rinse ability and skin finish, and is easy to carry while maintaining good makeup removal effects. It has a desired hardness to be adapted to the required equipment.

Cited document 2 discloses a solid anhydrous composition comprising a) at least one hydrophobic polymer chosen from the group consisting of ester terminated poly (ester-amide), and their mixtures; b) at least one hydrocarbon-based oil; and c) at least one non-ionic surfactant. The composition is useful for cleansing of the keratin materials and/or the makeup removal from the keratin materials.

Besides, in the prior art, the cleansing ability, user experience, makeup removal ability, and rinsing properties of anhydrous solid cleansing compositions have been studied to some extent. However, due to the presence of room temperature-solid components (such as wax) and room-temperature liquid components (such as liquid oil ingredients) in these solid compositions, there are stability problems caused by phase separation during production, transportation, storage and even in use, which have not received sufficient attention and reporting yet.

### CITED DOCUMENTS

Cited document 1: CN106852117A
Cited document 2: CN110062619A

### SUMMARY

### Problems to be Solved by the Disclosure

As mentioned above, although the cleansing ability, user experience, or washability of solid cleansing compositions have been investigated, the present disclosure has also found in long-term further study that although various room-temperature solid components and oil components are used in solid compositions to meet the above-mentioned use performance, this may also introduce stability problems.

For instance, during production, transportation, storage and even in use, the solid composition may be subjected to external influences such as vibration (e.g., resulting in an unconventional stable shape to stand) and temperature changes, and in which the solid components (including solid waxy components) and the liquid components such as oil may undergo phase separation, which may lead to a decrease in appearance, user experience, and even cleansing performance. Particularly, this phenomenon becomes particularly obvious after a large temperature difference has been applied to the solid composition for a period of time.

Therefore, the present disclosure intends to provide a topical composition for skin, which is a solid cleansing composition containing little or substantially no water. The composition not only has good cleansing performance, comfortable usability, and washability, but also has improved stability, which may avoid phase separation under the influence of external mechanical force or temperature changes.

### Solution to the Problems

Through long-term study by the inventors, it is found that the above-mentioned technical problems can be solved by the implementation of the following technical solutions.

[1] The present disclosure first provides a topical composition for skin, which comprises:
a) a solid particle, which is a porous particle having surface openings, and has an average particle size of primary particles of 0.5 to 20 μm;
b) at least one wax;
c) at least one carbon chain-based oil; and
d) at least one surfactant,
and wherein the composition optionally comprises water and has a water content of 5% or less based on the total mass of the composition.

[2] The composition according to [1], wherein the surface openings in the solid particle of component a) have an average pore diameter of 7.5 to 50 nm.

[3] The composition according to [1] or [2], wherein the solid particle of component a) is selected from one or more of silica, surface-modified silica, and organic polymer-based particles.

[4] The composition according to any one of [1] to [3], wherein the solid particle of component a) has an oil absorption of 300% or less and/or a specific surface area of 80 to 400 m²/g.

[5] The composition according to any one of [1] to [4], wherein the composition substantially does not comprise water.

[6] The composition according to any one of [1] to [5], wherein the wax of component b) is selected from one or more of natural plant wax and synthetic wax.

[7] The composition according to any one of [1] to [6], wherein in the composition, the content of component a) is 0.1 to 10%, the content of component b) is 1 to 30%, the content of component c is 50 to 95%, and the content of component d) is 3 to 30%, based on the total mass of the composition.

[8] Furthermore, the present disclosure provides a detergent, which comprises the composition according to any one of [1] to [7].

### Advantageous Effects of the Disclosure

By implementing the above-mentioned technical solutions, the present disclosure achieves the following technical effects:

1) the topical composition for skin provided by the present disclosure has good skin cleansing ability and excellent user experience, as well as satisfactory washability;

2) the topical composition for skin provided by the present disclosure may prevent deterioration of homogeneity caused by external mechanical force or temperature changes and has improved stability over time.

### BRIEF DESCRIPTION OF THE DRAWINGS

FIG. 1 is a temperature control chart for the stability test in the Examples of the present disclosure.

FIG. 2 is an illustration for placing the tested samples (upright and side elevation) during the evaluation test in the Examples of the present disclosure.

### DETAILED DESCRIPTION

Hereinafter, the present disclosure will be described in detail. The description of the to technical features described below is based on the representative embodiments and specific examples of the present disclosure, but the present disclosure is not limited to these embodiments and specific examples. It should be noted that:

In this specification, the numerical range expressed by “numerical value A to numerical value B” refers to the numerical range including the endpoints A and B.

In this specification, the numerical range expressed by “or more” or “or less” refers to the numerical range including the number mentioned herein.

In this specification, the meaning of “may” includes both the meaning of performing some processing and the meaning of not performing some processing.

In this specification, “optional” or “optionally” means using or not using certain factors such as substances, components, execution steps, or application conditions.

In this specification, “powder” and “particle” have the same physical meaning; “solid powdery particle” and “solid particle” also have the same physical meaning.

In this specification, the “room temperature” used means the indoor ambient temperature of “25° C.”.

In this specification, the units used are all international standard units, and unless otherwise stated, the “%” used all means weight percentage or mass percentage.

In this specification, “resin” and “rubber” have different meanings, and they are both distinguished by whether they have “elasticity,” i.e., usually, the former does not have apparent elasticity and the latter has apparent elasticity.

In the present disclosure, a “solid” composition refers to the form of the composition at 25° C., and “solid” refers to the composition whose hardness at 25° C. and atmospheric pressure (760 mmHg) is greater than or equal to 30 Nm⁻¹ when measured according to the following scheme. The specific test method may be a method described in the cited documents or the prior art.

In this specification, “containing substantially no water” means that in some specific embodiments of the present disclosure, no additional water is added at the time of mixing the components, i.e., the water that may be contained in the composition comes only from the inevitable and possible trace amount of moisture in the raw materials.

In this specification, “some specific/preferable embodiment”, “other specific/preferred embodiment”, “embodiment” and on the like mentioned herein mean that the specific elements (such as features, structures, properties and/or characteristics) described in connection with the embodiment is included in at least one embodiment described herein, and may or may not be present in other embodiments. Besides, it should be understood that the elements may be combined in various embodiments in any suitable manner.

The present disclosure relates to a topical composition for skin, which in some specific embodiments is a skin cleansing composition containing little or substantially no water.

The topical composition for skin according to the present disclosure mainly includes a solid particle which is a porous powder with surface openings (hereinafter, also referred to as “solid powdery particle”), a necessary waxy component, an oil component, and a surfactant. In some preferable embodiments, the water content is 5% or less based on the total mass of the composition.

(Solid Powdery Particle)

The topical composition for skin according to the present disclosure contains a solid powdery particle, and the solid powdery particle according to the present disclosure is a porous solid particle or porous solid powder having surface openings.

In the present disclosure, the porous solid particle or porous solid powder is present in the form of a primary particle in the topical composition for skin. In some specific embodiments, the porous solid particle or porous solid powder present in the form of a primary particle accounts for 90% or more, preferably 95% or more, and further preferably 98% or more of the total mass of all particles or powder.

The “primary particle” described in the present disclosure refers to a particle present in the state of a single particle, which is distinguished from a “secondary particle” in the existing state. The latter refers to aggregated particles formed by gathering several single particles together because of high surface energy due to the significantly small particle size of solid particles, such as aerogels.

In some specific embodiments of the present disclosure, the primary particle according to the present disclosure has an average primary particle size (D50) of 0.5 to 20 μm, preferably 3 to 18 μm, and further preferably 4 to 15 μm. If the average particle size of the primary particles is excessively small, it may cause the agglomeration of the primary particles to form secondary particles and thus have a tendency to deteriorate the stability of the composition described in the present disclosure. If the average particle size of the primary particles is excessively large, it may adversely affect the comfort of the topical composition for skin of the present disclosure during use, such as excessive particle sensation or excessive friction sensation.

The above-mentioned porous solid particle or powder according to the present disclosure may be selected from silica particles having an opening structure on the surface, surface-modified silica particles, organic polymer-based particles, or a mixture thereof. Preferably, the porous solid particles or powder according to the present disclosure are preferably silica particles having an opening structure on the surface thereof in consideration of user experience, thermal expansion and contraction resistance of materials, and safety. In addition, in some specific embodiments, the porous solid particle or powder according to the present disclosure may be hydrophobic particles or powder.

Further, the surface-modified silica may be silica particles with surfaces modified by resin or rubber, such as silicone resin or silicone rubber; the organic polymer-based particles may be selected from particles made of crosslinked or uncrosslinked organic polymer materials, and examples thereof include particles formed of (meth) acrylate resin, polyamide resin (nylon powder), polyethylene, polystyrene, copolymer resin of styrene and (meth) acrylic acid, benzoguanamine resin, polytetrafluoroethylene, cellulose, organic silicone resin and silicone rubber, etc., or a composite (such as a core-shell type) particles formed of two or more thereof.

For the opening structure on the surface of the porous solid particle or powder according to the present disclosure, the “opening” means that there are holes communicating with the outside on the surface of the particle or powder.

In some specific embodiments, these openings have an average pore diameter of 7.5 nm or more, or 8 nm or more, or 10 nm or more, or 12 nm or more; in some specific embodiments, the upper limit of the average pore diameter of the openings is 50 nm or less, or 30 nm or less, or 20 nm or less. Further, in some preferable embodiment of the present disclosure, the average pore diameter of the openings is preferably 7.5 to 15 nm.

In the present disclosure, the average pore diameter of the above-mentioned surface openings may be determined by conventional methods. Typically, the determination may be made by means of the standard of gas adsorption analytical tests carried out by nitrogen adsorption at the liquid nitrogen temperature, and the obtained data is processed by Barret, Joyner, and Halenda method (abbreviated as BJH method) to get the final determined value.

In the present disclosure, the specific surface area of the porous solid particles, in some specific embodiments, may be 400 m²/g or less, preferably 80 to 400 m²/g or 100 to 400 m²/g, further preferably 200 to 350 m²/g. In the present disclosure, the specific surface area may be determined by the BET (Brunauer-Emmett-Teller) nitrogen adsorption method described in Journal of the American Chemical Society, Volume 60, Page 309, February 1938 and corresponding to the international standard ISO 5794/1 (Appendix D). The BET specific surface area corresponds to the total specific surface area of the particles to be tested.

Besides, in the present disclosure, the oil absorption of the porous solid particle is not particularly limited, and it is related to the specific surface area and particle size. However, from the viewpoint of the stability of the topical composition for skin according to the present disclosure, the porous solid particle according to the present disclosure has an oil absorption of 300% or less, preferably 50 to 250% or 100 to 250%. The oil absorption may be measured by conventional techniques, for example, according to the wet point method or method for determining the oil uptake of a powder described in Standard NF T 30-022.

In the present disclosure, it is considered that the use of porous solid particles or solid powder with the features defined above is beneficial to prevent stability deterioration caused by phase separation when mixed with other components in the topical composition for skin according to the present disclosure.

According to the study of the present disclosure, it is speculated that the above-mentioned effect may be caused by the following mechanism. In the topical composition for skin according to the present disclosure, the waxy component is a main room-temperature solid component at while mixing with oily components, solid particles, surfactant, and the like. During the mixing process, the oily component may enter the surface openings of the solid particles, i.e., the surface opening structure functions to anchor the entering oily components. As a result, the finally formed topical composition for skin may prevent separation and precipitation of the oily components from the solid phase of the topical composition for skin even after experiencing external mechanical force stimulation (such as vibration during transportation or use) or temperature change stimulation (such as temperature change due to storage conditions or regional and seasonal differences), thereby improving the overall stability of the topical composition for skin according to the present disclosure and maintaining the original usability and cleansing ability. Moreover, such anchoring effect may be well reflected even when the content of the porous solid particles is 10% or less.

(Waxy Component)

The topical composition for skin of the present disclosure comprises a waxy component.

In the present disclosure, the waxy component refers to a lipid compound component that presents as a solid at room temperature.

In some specific embodiments of the present disclosure, the waxy component has a melting point of 30° C. or more, preferably has a melting point of 50° C. or more, more preferably has a melting point of 60° C. or more, and further preferably has a melting point of 65° C. or more. The melting point described in the present disclosure may be determined, for example, by the temperature of the maximum endothermic peak observed by thermal analysis (DSC) described in the standard ISO 11357-3; 1999. Specifically, the melting point of the waxy component may be determined using a differential scanning calorimeter (DSC), such as the calorimeter sold under the name MDSC 2920 by TA Instruments.

The waxy component which may be used in the topical composition for skin according to the present disclosure may be selected from natural animal and plant waxes or synthetic waxes. Specifically, the waxy component may be selected from waxes of animal, plant, mineral or synthetic origin that are solid at room temperature, and mixtures thereof, and preferably, it may be one or more of natural plant waxes and synthetic waxes.

Specific examples of the waxy component which may be used in the present disclosure include hydrocarbon-based waxes such as beeswax, lanolin wax, insect wax, rice bran wax, carnauba wax, candelilla wax, ouricury wax, esparto grass wax, berry wax, shellac wax, Japan wax, and sumach wax; montan wax, orange wax, lemon wax, microcrystalline wax, ceresin wax, polyethylene wax, polymethylene wax, 12-hydroxystearic acid, glyceryl trihydroxystearate, waxes, and wax-like copolymers obtained by Fischer-Tropsch synthesis, as well as esters thereof and mixtures thereof. Examples may further include waxes obtained by catalytic hydrogenation of animal oil or vegetable oil containing straight chain or branched C8-C32 fatty chains. Among these waxes, the isomerized jojoba oil may be particularly mentioned, such as trans-isomerized partially hydrogenated jojoba oil, hydrogenated sunflower oil, hydrogenated castor oil, hydrogenated coconut oil, hydrogenated lanolin oil manufactured or sold by Desert Whale Company under the commercial reference name, and bis(1,1,1-trimethylolpropane) tetrastearate sold by Heterene Company under the name Hest.

In some specific embodiments of the present disclosure, waxes (such as the product named Phytowax ricin by Sophim) obtained by hydrogenation of castor oil esterified with cetyl alcohol, C20-C40 alkyl (hydroxy-stearyloxy) stearate (such as the products named Kester Wax K 82 Hydroxypolyester K 82 and Kester Wax K 80 by Koster Keunen), and linear hydroxylated C18-C24 fatty acids (such as the 12-hydroxystearic acid named 12-Hydroxystearic Acid Premium Grade 12H-P by Thai Kawaken) may be used.

In some specific embodiments of the present disclosure, the topical composition for skin according to the present disclosure comprises at least one waxy component which has a melting point greater than or equal to 60° C. These waxes may be selected from carnauba wax, ceresin, microcrystalline wax, 12-hydroxystearic acid, polyethylene wax (such as those sold by New Phase Technologies under the name Performalene 500L Polyethylene or Performalene 400L Polyethylene, or Asensa SC 211 from Honeywell), polymethylene wax (such as the product sold by Cirebelle under the reference name Cirebelle 108), beeswax, candelilla wax, hydroxy octacosanol hydroxystearate, hydrogenated castor oil, hydrogenated jojoba oil, rice bran wax, polyglycerolated beeswax, octacosanol stearate, ceresin wax, C20-C40 alkyl (hydroxystearyloxy) stearate wax, 12-hydroxystearic acid, polyvinyl alcohol wax, Fischer-Tropsch wax, wax obtained by hydrogenation of castor oil esterified with cetyl alcohol, ouricury wax, montan wax, glyceryl trihydroxystearate named Trihydroxystearin from INCI (for example, sold by Elementis under the name Thixcin R), and mixtures thereof.

In some specific embodiments of the present disclosure, the topical composition for skin according to the present disclosure comprises at least one wax, which has a melting point greater than or equal to 65° C. and may be selected from carnauba wax, ceresin, microcrystalline wax, 12-hydroxystearic acid, polymethylene wax (for example, the product sold by Cirebelle under the reference name Cirebelle 108), candelilla wax, hydroxyoctacosanol hydroxystearate, hydrogenated castor oil, hydrogenated jojoba oil, rice bran wax, polyglycerolated beeswax, octacosanol stearate, paraffin wax, C20-C40 alkyl (hydroxystearyloxy) stearate wax, polyvinyl alcohol wax, Fischer-Tropsch wax, wax obtained by hydrogenation of castor oil esterified with cetyl alcohol, ouricury wax, montan wax, glyceryl trihydroxystearate named Trihydroxystearin in INCI (for example, sold by Elementis under the name Thixcin R), and mixtures thereof.

One or more of the above-mentioned waxy components may be used, including, for example, a mixture of waxy components with the same melting point or near the same melting point, or a mixture of waxy components with different melting points.

In some more preferable embodiments of the present disclosure, the composition according to the present disclosure comprises at least one waxy component having a melting point greater than or equal to 65° C., which is selected from carnauba wax, ceresin, microcrystalline wax, polymethylene wax, 12-hydroxystearic acid, candelilla wax, Fischer-Tropsch wax, hydrogenated jojoba oil and glyceryl trihydroxystearate, and mixtures thereof

(Carbon Chain-Based Oil)

In the present disclosure, the topical composition for skin comprises an oil substance, which includes at least one carbon chain-based oil.

In the present disclosure, said “hydrocarbon-based” means that the molecular backbone structure of the oil is mainly formed based on carbon-carbon bonds, and such backbone structure includes a main chain structure and/or a branched chain structure, thereby distinguishing the oil from silicon chain-based oils.

In the present disclosure, the carbon chain-based oil is generally an oil that has a liquid form at room temperature and 1 atmospheric pressure, and such oil may be volatile oil or nonvolatile oil, and preferably, nonvolatile liquid oil is used. The term “nonvolatile liquid oil” in this specification means liquid oil which does not show volatility at normal temperature (25° C.)/atmospheric pressure (1 atmosphere) (for example, comprising an oil component having a boiling point of about 200° C. or more at atmospheric pressure) and has fluidity and is not solid at normal temperature/atmospheric pressure.

In the present disclosure, the carbon chain-based oil may be liquid oil from petrochemical industry or liquid oil from animals or plants, and may be specifically selected from hydrocarbon oil, vegetable oil, mineral oil, ester oil, ether oil, etc.

Specific examples include liquid oils such as linseed oil, camellia oil, macadamia oil, corn oil, olive oil, avocado oil, camellia oil, castor oil, safflower oil, almond oil, cinnamon oil, jojoba oil, grape oil, sunflower oil, wheat germ oil, rice germ oil, rice bran oil, cottonseed oil, soybean oil, peanut oil, camellia oil, evening primrose oil, egg yolk oil, liver oil, triglycerin, tricaprylin, citric acid ester and triisopalmitic glyceride; octanoic esters such as cetyl octanoate and glyceryl tricaprylate; isooctanoic acid esters such as glyceryl tri(2-ethylhexanoate), pentaerythritol tetra(2-ethylhexanoate), and cetyl (2-ethylhexanoate); laurates such as hexyl laurate; myristic acid esters such as isopropyl myristate and octyl dodecyl myristate; palmitates such as octyl palmitate; stearic acid esters such as isocetyl stearate; isostearate such as isopropyl isostearate; isopalmitates such as octyl isopalmitate; oleates such as isodecyl oleate; adipic acid diesters such as diisopropyl adipate; sebacate diesters such as diethyl sebacate; ester oils such as diisostearyl malate; linear/branched hydrocarbon oil or polyolefin oil with 8-30 carbon atoms, such as isododecane, isohexadecane, hydrogenated polyisobutylene, liquid paraffin, and squalane; higher fatty alcohols, including linear alcohols (such as lauryl alcohol, cetyl alcohol, stearyl alcohol, behenyl alcohol, myristyl alcohol, oleyl alcohol and cetearyl alcohol) and branched alcohols (such as monostearyl glycerol ether (shark liver alcohol), 2-decyl tetradecanol, lanolin alcohol, cholesterol, phytosterol, hexyldodecanol, isostearyl alcohol and octyldodecanol), etc.; higher fatty acids, such as lauric acid, myristic acid, palmitic acid, stearic acid, behenic acid, oleic acid, undecenoic acid, tall oil fatty acid, isostearic acid, linolic acid, linoleic acid, eicosapentaenoic acid (EPA) and docosahexaenoic acid (DHA), etc.; polymer ether oils of ethers or polyalkylenes with 10 to 40 carbon atoms, such as polyoxybutylene glycol and polyoxypropylene glycol.

One of or a mixture of more of the above-mentioned carbon chain-based oils may be used. In some preferable embodiments of the present disclosure, polar oils are preferably used as the above-mentioned carbon chain-based oil from the viewpoint of usability and stability. Moreover, in some specific embodiments, the content of polar oils in the carbon chain-based oil is 80% or more, preferably 85% or more, and further preferably 90% or more.

In addition, under the premise of not affecting the technical effect of the present disclosure, silicon chain-based oils, such as volatile or nonvolatile silicone oils, may also be used in the oily substance of the present disclosure.

Volatile silicone oils are silicone oils that are volatile at room temperature and have been used in cosmetics, for example, volatile linear silicone oils (volatile polydimethylsiloxane) and volatile cyclic silicone oil (volatile cyclomethylsiloxane). As the volatile polydimethylsiloxane, low-viscosity dimethyl polysiloxanes such as decamethyltetrasiloxane may be used. Examples of commercial products include KF-96L-1.5cs, KF-96L-2cs (both manufactured by Shin-Etsu Chemical Co., Ltd.), etc. Examples of volatile cyclomethylsiloxane include decamethylcyclopentasiloxane (D5), etc.

In some specific embodiments of the present disclosure, in the topical composition for skin of the present disclosure, the content of the carbon chain-based oil is 80% or more, preferably 85% or more, and further preferably 90% or more based on the total mass of the oily substance.

(Surfactant)

A surfactant is included in the topical composition for skin of the present disclosure. There is no particular limitation on the types of the surfactant, and one or more of anionic surfactants, cationic surfactants, amphoteric surfactants, and nonionic surfactants commonly used in the field may be used.

Examples of anionic surfactants that may be used in the present disclosure include: fatty acid soaps (such as sodium laurate and sodium palmitate); higher alkyl sulfate salts (such as sodium lauryl sulfate and potassium lauryl sulfate); alkyl ether sulfate salts (such as POE-triethanolamine lauryl sulfate and POE-sodium lauryl sulfate); N-acyl sarcosine (such as sodium N-lauroyl sarcosine); higher fatty acid amide sulfonates (such as sodium N-myristoyl N-methyl taurate, sodium cocoyl methyl taurate and sodium lauryl methyl taurate); phosphate salts (such as POE-oil-based ether sodium phosphate and POE stearyl ether sodium phosphate); sulfosuccinates (such as sodium di-2-ethylhexyl sulfosuccinate, sodium mono-lauroyl monoethanolamide polyoxyethylene sulfosuccinate and sodium lauryl polypropylene glycol sulfosuccinate); alkylbenzene sulfonates (such as linear dodecylbenzene sulfonate, triethanolamine linear dodecylbenzene sulfonate and linear dodecylbenzene sulfonic acid); higher fatty acid ester sulfate (such as sodium glyceryl sulfate of hydrogenated coconut oil fatty acid); N-acyl glutamate (such as monosodium N-lauroyl glutamate, disodium N-stearoyl glutamate and sodium N-myristoyl-L-glutamate); sulfated oil (such as turkey red oil); POE-alkyl ether carboxylic acid; POE-alkyl aryl ether carboxylate; α-olefin sulfonate; higher fatty acid ester sulfonate; secondary alcohol sulfate; higher fatty acid alkyl amide sulfate; sodium lauroyl monoethanolamine succinate; bistriethanolamine N-palmitoyl aspartate; and sodium caseinate.

Examples of cationic surfactants that may be used in the present disclosure include: alkyl trimethyl ammonium salts (such as stearyl trimethyl ammonium chloride and lauryl trimethyl ammonium chloride); alkyl pyridinium salts (such as cetylpyridinium chloride); disstearyl dimethyl ammonium chloride dialkyl dimethyl ammonium salt; poly (N,N′-dimethyl-3-methylene piperidinium) chloride; alkyl quaternary ammonium salt; alkyl dimethyl benzyl ammonium salt; alkyl isoquinoline onium salt; dialkyl morpholine salt; POE alkylamine; alkyl amine salt; polyamine fatty acid derivatives; pentanol fatty acid derivatives; benzalkonium chloride; and benzethonium chloride.

Examples of amphoteric surfactants that may be used in the present disclosure include: imidazoline type amphoteric surfactants (such as 2-undecyl-N,N,N-(hydroxyethyl carboxymethyl)-2-imidazoline sodium salt and 2-cocoyl-2-imidazoline hydroxide 1-carboxyethoxy disodium salt, etc.); and betaine-type surfactants (such as 2-heptadecyl-n-carboxymethyl-n-hydroxyethyl imidazoline betaine, dodecyl dimethylaminoacetic acid betaine, alkyl betaine, amidobetaine, and sulfobetaine, etc.).

Examples of nonionic surfactants that may be used in the present disclosure include hydrophilic nonionic surfactants and lipophilic surfactants.

Examples of the hydrophilic surfactant include: polyglycerol fatty acid esters (such as hexaglycerol monolaurate (HLB 14.5), hexaglycerol monomyristate (HLB 11), hexaglycerol monostearate (HLB 9.0), hexaglycerol monooleate (HLB 9.0), decaglycerol monolaurate (HLB 15.5), decaglycerol monomyristate (HLB 14.0), and decaglycerol monostearate (HLB 12.0), decaglycerol monoisostearate (HLB 12.0), decaglycerol monooleate (HLB 12.0), decaglycerol distearate (HLB 9.5), and decaglycerol diisosearate (HLB 10.0)); polyoxyethylene glycerol fatty acid esters (such as polyoxyethylene (“POE”) (5) glycerol monostearate (HLB 9.5), POE(15) glycerol monostearate (HLB 13.5), POE(5) glycerol monooleate (HLB 9.5), and POE(15) glycerol monooleate (HLB 14.5)); polyoxyethylene sorbitan fatty acid esters (such as POE(20) sorbitan monostearate (HLB 16.9), POE(20) sorbitan monopalmitate (HLB 15.6), POE(20) sorbitan monostearate (HLB 14.9), POE(6) sorbitan monostearate (HLB9.5), POE(20) sorbitan tristearate (HLB 10.5), POE(20) sorbitan monoisostearate (HLB 15.0), POE(20) sorbitan monooleate (HLB 15.0), POE(6) sorbitan monooleate (HLB 10.0) and POE(20) sorbitan triooleate (HLB 11.0); polyoxyethylene sorbitol fatty acid esters (such as POE(6) sorbitol monolaurate (HLB 15.5), POE(60) sorbitol tetrastearate (HLB 13.0), POE(30) sorbitol tetraoleate (HLB11.5), POE(40) sorbitol tetraoleate (HLB 12.5) and POE(60) sorbitol tetraoleate (HLB114.0)); polyoxyethylene lanolin/lanolin alcohol/beeswax derivatives (such as POE(10) lanolin (HLB 12.0), POE(20) lanolin (HLB 13.0), POE(30) lanolin (HLB 15.0), POE(5) lanolin alcohol (HLB 12.5), POE(10) lanolin alcohol (HLB 15.5), POE(20) lanolin alcohol (HLB 16.0), POE(40) lanolin alcohol (HLB 17.0) and POE(20) sorbitol beeswax (HLB 9.5); polyoxyethylene castor oil/hydrogenated oil (such as POE(20) castor oil (HLB 10.5), POE(40) castor oil (HLB 12.5), POE(50) castor oil (HLB 14.0), POE(60) castor oil (HLB 14.0), POE(20) hydrogenated castor oil (HLB10.5), POE(30) hydrogenated castor oil (HLB11.0), POE(40) hydrogenated castor oil (HLB 13.5), POE(60) hydrogenated castor oil (HLB 14.0), POE(80) hydrogenated castor oil (HLB 16.5), and POE(100) hydrogenated castor oil (HLB16.5); polyoxyethylene sterol/hydrogenated sterol (such as POE(5) phytosterol (HLB 9.5), POE(10) phytosterol (HLB 12.5), POE(20) phytosterol (HLB 15.5), POE(30) phytosterol (HLB 18.0), POE(25) phytosterol (HLB 14.5), and POE(30) cholestanol (HLB17.0)); polyoxyethylene alkyl ether (such as POE(2) lauryl ether (HLB 9.5), POE(4.2) lauryl ether (HLB 11.5), POE(9) lauryl ether (HLB 14.5), POE(5.5) cetyl ether (HLB 10.5), POE(7) cetyl ether (HLB 11.5), POE(10) cetyl ether (HLB 13.5), POE(15) cetyl ether (HLB 15.5), POE(20) cetyl ether (HLB 17.0), POE(23) cetyl ether (HLB 18.0), POE(4) stearyl ether (HLB 9.0), POE(20) stearyl ether (HLB 18.0), POE(7) oil-based ether (HLB 10.5), POE(10) oil-based ether (HLB 14.5), POE(15) oil-based ether (HLB 16.0), POE(20) oil-based ether (HLB 17.0), POE(50) oil-based ether (HLB 18.0), POE(10) docosanyl ether (HLB 10.0), POE(20) docosanyl ether (HLB 16.5), POE(30) docosanyl ether (HLB 18.0), POE(2)(C12-15) alkyl ether (HLB 9.0), POE(4)(C12-15) alkyl ether (HLB 10.5), POE(10)(C12-15) alkyl ether (HLB 15.5), POE(5) secondary alkyl ether (HLB 10.5), POE(7) secondary alkyl ether (HLB 12.0), POE(9) alkyl ether (HLB 13.5) and POE(12) alkyl ether (HLB14.5)); polyoxyethylene polyoxypropylene alkyl ether (such as POE(1) polyoxypropylene (“POP”)(4) cetyl ether (HLB 9.5), POE(10)POP(4) cetyl ether (HLB 10.5), POE(20)POP(8) cetyl ether (HLB 12.5), POE(20)POP(6) decyl tetradecyl ether (HLB 11.0) and POE(30)POP(6) decyl tetradecyl ether (HLB12.0)); polyethylene glycol fatty acid ester (such as polyethylene glycol (“PEG”)(10) (HLB12.5), PEG(10) monostearate (HLB 11.0), PEG(25) monostearate (HLB 15.0), PEG(40) monostearate (HLB 17.5), PEG(45) monostearate (HLB 18.0), PEG(55) monostearate (HLB 18.0), PEG(10) monooleate (HLB 11.0), PEG distearate (HLB 16.5) and PEG distearate (HLB 9.5)); and (III) polyoxyethylene glyceryl isostearate (such as PEG(8) glyceryl isostearate (HLB 10.0), PEG(10) glyceryl isostearate (HLB 10.0), PEG(15) glyceryl isostearate (HLB 12.0), PEG(20) glyceryl isostearate (HLB 13.0), PEG(25) glyceryl isostearate (HLB 14.0), PEG(30) glyceryl isostearate (HLB 15.0), PEG(40) glyceryl isostearate (HLB 15.0), PEG(50) glyceryl isostearate (HLB 16.0) and PEG(60) glyceryl isostearate (HLB 16.0)).

Examples of lipophilic surfactants include POE(2) stearyl ether (HLB 4.0), self-emulsifying propylene glycol monostearate (HLB 4.0), glyceryl myristate (HLB 3.5), glyceryl monostearate (HLB 4.0), self-emulsifying glyceryl monostearate (HLB 4.0), glyceryl monoisostearate (HLB 4.0), glyceryl monooleate (HLB 2.5), hexaglycerol tristearate (HLB 2.5), decaglycerol pentastearate (HLB 3.5), decaglycerol pentaisostearate (HLB 3.5), decaglycerol pentaoleate (HLB 3.5), sorbitan monostearate (HLB 4.7), sorbitan tristearate (HLB 2.1), sorbitan monoisostearate (HLB 5.0), sorbitan sesquistearate (HLB 4.5), sorbitan monooleate (HLB 4.3), POE(6) sorbitol hexastearate (HLB 3.0), POE(3) castor oil (HLB 3.0), PEG(2) monostearate (HLB 4.0), ethylene glycol monostearate (HLB 3.5), and PEG(2) stearate (HLB 4.5).

In some preferable embodiments of the present disclosure, from the viewpoint of user experience and stability, it is preferable to use one or more nonionic surfactants having HLB value of 4.5 to 20, preferably 5 to 15.

(Topical Composition for Skin)

The topical composition for skin of the present application may be obtained by uniformly mixing the above-mentioned components. The manner of mixing is not particularly limited. The components may be mixed simultaneously, or a few components may be mixed together before being mixed with other components. In some preferable embodiment of the present disclosure, the above-mentioned solid powdery particles may be mixed with at least part of the carbon chain-based oil, and then mixed with other components to obtain the topical composition for skin of the present disclosure.

Besides, regarding the mixing temperature, in some specific embodiments, the mixing may be carried out within the range of 30 to 70° C.

The amount of the solid powdery particles in the present disclosure may be 0.1 to 10%, preferably 0.3 to 8%, and further preferably 0.5 to 5% based on the total mass of the topical composition for skin.

The amount of wax in the present disclosure may be 1 to 30%, preferably 2 to 20%, further preferably 3 to 15%, and even more preferably 3 to 10% based on the total mass of the topical composition for skin.

The amount of the carbon chain-based oil in the present disclosure may be 50 to 95%, preferably 55 to 90%, and further preferably 60 to 85% based on the total mass of the topical composition for skin.

The amount of the surfactant in the present disclosure may be 3 to 30%, preferably 5 to 28%, and further preferably 10 to 25% based on the total mass of the topical composition for skin.

In some specific embodiments of the present disclosure, the water content (based on the total mass of the composition) in the topical composition for skin of the present disclosure is 5% or less, preferably 3% or less, further preferably 1% or less, and even more preferably 0.5% or less. In some preferable embodiments, the topical composition for skin of the present disclosure contains substantially no water; or there is no additional water added at the time of mixing the composition of the present disclosure, in which case the water in the composition of the present disclosure is only derived from the residual water unavoidably present in each component.

Furthermore, in some specific embodiments of the present disclosure, the total amount of components a) to d) may be 85% or more, preferably 90% or more, and further preferably 95% or more based on the total mass of the topical composition for skin.

The hardness of the topical composition for skin of the present disclosure is not particularly required. In some specific embodiments, the topical composition for skin of the present disclosure has a hardness of 40 N/m or more, preferably a hardness of 50 to 300 N/m. Besides, the topical composition for skin of the present disclosure may or may not have a fixed shape, may be directly applied to human body or skin, and may also be loaded on other carriers or applied to human body or skin with the assistance of other carriers.

Besides, in addition to the above-mentioned components, the composition of the present disclosure may also comprise various auxiliary ingredients suitable for use in cosmetic compositions, as long as they do not interfere with the technical effects of the present disclosure. Such auxiliary ingredients include, but are not limited to, preservatives, antioxidants, complexing agents, pH regulators (acidic or alkaline), perfumes, other fillers, bactericides, odor absorbers, colorants (pigments and dyes), film-forming polymers, thickeners and/or gelling agents, active ingredients (such as sodium hyaluronate, plant extracts such as madecassoside) and the like. In some other specific embodiments, the topical composition for skin of the present disclosure is a solid having self-forming and self-supporting properties, so it is unnecessary to include (water-insoluble) artificial or natural polymer-based support carriers, such as mesh, filamentous, and fabric-like carriers.

In addition, the topical composition for skin of the present disclosure may be prepared into various cosmetic products according to needs, such as cream, paste, solid or gel cosmetic products with fixed appearance. In some preferable embodiments, the topical composition for skin of the present disclosure may be used for preparing products for cleansing human body or skin, more preferably for preparing products for cleansing agent used for purposes such as makeup removal.

### EXAMPLES

The technical solution of the present disclosure will be described below with reference to specific examples.

<Raw Materials>

In the present disclosure, the names and sources of the raw materials were shown in Table 1 below.

TABLE 1

| Names of raw materials | Sources of raw materials |
|---|---|
| Mineral oil | CHINA PETROCHEMICAL CO. |
| | HANG ZHOU |
| Cetyl ethyl hexanoate | NIPPON FINE CHEMICAL CO., LTD. |
| Glyceryl tri(ethyl hexanoate) | NIPPON FINE CHEMICAL CO., LTD. |
| PEG-10 isostearate | NIHON EMULSION COMPANY, LTD. |
| PEG-20 glyceryl triisostearate | NIHON EMULSION CO., LTD. |
| Synthetic wax | JAPAN NATURAL PRODUCTS INC. |
| Dimethyl silylated silica | NIHON AEROSIL |
| Silica | AGC SI-TECH. CO., LTD. |
| Methyl methacrylate | SEKISUI PLASTICS CO., Ltd. |
| crosslinked polymer | |
| Divinyl polydimethylsiloxane/ | SHIN-ETSU CHEMICAL CO., LTD. |
| polydimethylsiloxane | |
| crosslinked polymer | |

Table 2 below showed physical property data of the solid particles in Table 1.

TABLE 2

| | | | | Average pore | |
|---|---|---|---|---|---|
| | | | | size (nm) of | |
| | | | | openings on | Specific |
| | | Oil | Average particle | primary | surface |
| | | absorption | size of primary | particle | area |
| Solid particles | | (%) | particles (μm) | surface | (m²/g) |
| Dimethyl silylated | AEROSIL R972 | 450 | about 0.016 | no opening on | 126.4 |
| silica | (aerogel) | | | primary | |
| | | | | particle surface | |
| Silica | SUNSPHERE | 151 | 4.5 to 5.5 | 11.13 | 319.6 |
| | L-51S | | | | |
| Methyl methacrylate | TECHPOLYMER | 169 | about 8 | 12.25 | 138.3 |
| crosslinked polymer | MBP-8HP | | | | |
| Divinyl | KSP-100 | 71 | 5 | 8.15 | 4.9 |
| polydimethylsiloxane/ | | | | | |
| polydimethylsiloxane | | | | | |
| crosslinked polymer | | | | | |

<Preparation Method>

Preparation of Raw-Material Mixture:

Component A: a wax was put into part of hydrocarbon-based oil and heated to 90+/−2° C. until it was completely melted;

Component B: the solid particles were mixed with the remaining oil, vigorously stirred to be completely dispersed, and then component A was added to the mixture, into which the other raw materials were added in turn at a temperature controlled at 85+/−2° C. After mixing, the mixture was filtered and discharged, followed by collection and storage.

Filling of the Raw-Material Mixture:

The raw-material mixture was charged into a kettle, heated to 82+/−2° C. Filling was carried out after the raw-material mixture was fully melted. After the filling was completed, the semi-finished product was cooled to obtain a cylindrical cooled product, which was packed into boxes and stored at room temperature.

<Test Method>

Usability Evaluation:

The skin (face) was cleaned by using the solids obtained in the following Examples and Comparative Examples, and it was tested whether greasy feeling was generated after rinsing with water (average data from 20 individuals).

Stability Evaluation:

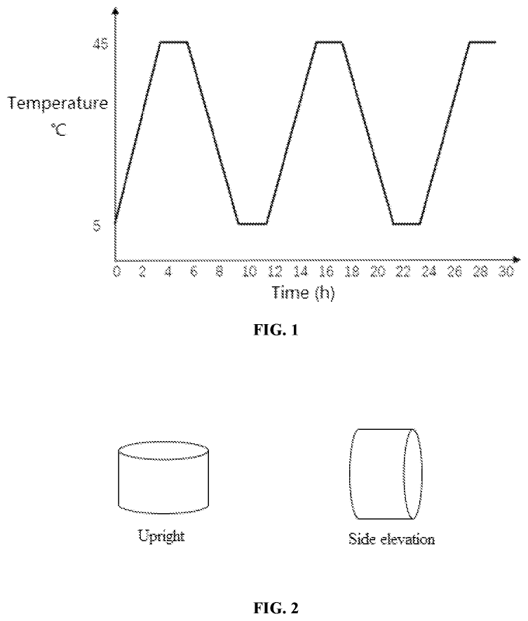

A test sample was placed in a manner of side elevation in an oven which was set at a temperature of 5 to 45° C., and the test was performed two cycles a day, wherein the sample were kept at 5° C. and 45° C. for 2 hours. The temperature change treatment was carried out in accordance with the thermostat setting curve as shown by the temperature control program shown in FIG. 1, for a total of 28 cycles in 14 days, and then it was observed whether there was any oil penetration on the surface of the test sample.

By standing the cylinder in a manner of side elevation (instead of upright, see FIG. 2) and performing temperature change treatment, the system stability under an abnormal storage state coupled with temperature change that may occur during transportation and storage was simulated.

### Examples 1 to 3, Comparative Examples 1 and 2

The respective components in Table 1 were prepared into compositions according to the above-mentioned method and tested.

TABLE 3

| | | Comparative | Comparative | Example | Example | Example |
|---|---|---|---|---|---|---|
| | | Example 1 | Example 2 | 1 | 2 | 3 |
| Oil | mineral oil | 15.15 | 13.15 | 13.15 | 13.15 | 13.15 |
| | Glyceryl tri(ethyl | 30 | 30 | 30 | 30 | 30 |
| | hexanoate) | | | | | |
| | cetyl ethyl | 30 | 30 | 30 | 30 | 30 |
| | hexanoate | | | | | |
| Surfactant | PEG-10 isostearate | 5 | 5 | 5 | 5 | 5 |
| | PEG-20 glyceryl | 13 | 13 | 13 | 13 | 13 |
| | triisostearate | | | | | |
| Wax | synthetic wax | 6.5 | 6.5 | 6.5 | 6.5 | 6.5 |
| Solid | dimethyl silylated | — | 2 | — | — | — |
| particles | silica | | | | | |
| | silica | — | — | 2 | — | — |
| | methyl | — | — | — | 2 | — |
| | methacrylate | | | | | |
| | crosslinked polymer | | | | | |
| | divinyl | — | — | — | — | 2 |
| | polydimethylsilox- | | | | | |
| | ane/polydimethyl- | | | | | |
| | siloxane crosslinked | | | | | |
| | polymer | | | | | |
| Antioxidant | butylated hydroxy | 0.05 | 0.05 | 0.05 | 0.05 | 0.05 |
| | toluene | | | | | |
| Preservative | dipropylene glycol | 0.3 | 0.3 | 0.3 | 0.3 | 0.3 |

| | | | | | |
|---|---|---|---|---|---|
| Total amount | 100 | 100 | 100 | 100 | 100 |

Evaluation

| | | | | | |
|---|---|---|---|---|---|
| Usability | Yes | A little | No | No | No |
| Stability | Oil | Oil | No | No | No |
| | penetrated | penetrated | | | |

It should be noted that although specific examples are provided to illustrate the technical solution of the present disclosure, those skilled in the art would understand that the present disclosure should not be limited thereto.

Although the embodiments of the present disclosure have been described above, it will be appreciated that the above descriptions are merely exemplary, but not exhaustive; and that the disclosed embodiments are not limiting. A number of variations and modifications may apparent to those skilled in the art without departing from the scopes and spirits of the described embodiments. The terms used in the present disclosure are selected to provide the best explanation on the principles and practical applications of the embodiments, and the technical improvements to the arts on market, or to make the embodiments described herein understandable to those skilled in the art.

### INDUSTRIAL AVAILABILITY

The topical composition for skin of the present disclosure may be industrially prepared and applied.

1. A topical composition for skin, which comprises:
a) a solid particle, which is a porous particle having surface openings, and has an average particle size of primary particles of 0.5 to 20 μm;
b) at least one wax;
c) at least one carbon chain-based oil; and
d) at least one surfactant,
and wherein the composition optionally comprises water and has a water content of 5% or less based on the total mass of the composition.

2. The composition according to claim 1, wherein the surface openings in the solid particle of component a) have an average pore diameter of 7.5 to 50 nm.

3. The composition according to claim 1, wherein the solid particle of component a) is selected from one or more of silica, surface-modified silica, and organic polymer-based particles.

4. The composition according to claim 1, wherein the solid particle of component a) has an oil absorption of 300% or less and/or a specific surface area of 80 to 400 m²/g.

5. The composition according to claim 1, wherein the composition substantially does not comprise water.

6. The composition according to claim 1, wherein the wax of component b) is selected from one or more of natural plant wax and synthetic wax.

7. The composition according to claim 1, wherein in the composition, the content of component a) is 0.1 to 10%, the content of component b) is 1 to 30%, the content of component c is 50 to 95%, and the content of component d) is 3 to 30%, based on the total mass of the composition.

8. A detergent, which comprises the composition according to claim 1.
